(19) United States
(12) Patent Application Publication
(10) Pub. No.: US 2024/0172727 A1
(43) Pub. Date: May 30, 2024

(54) Microscope Systems, Software, and Methods for Performing Neural Experiments on Living and/or Moving Subjects

(71) Applicant: Microbrightfield, LLC, Williston, VT (US)

(72) Inventors: Jacob R. Glaser, Shelburne, VT (US); David Denu, Essex Junction, VT (US); Peter Lang, Cornelius, NC (US)

(21) Appl. No.: 18/521,326

(22) Filed: Nov. 28, 2023

Related U.S. Application Data
(60) Provisional application No. 63/428,306, filed on Nov. 28, 2022.

(51) Int. Cl. A01K 29/00; A61B 5/00; A61B 5/103; A61B 5/11; A61B 18/20; A61B 90/20

(52) U.S. Cl. CPC A01K 29/005; A61B 5/0071; A61B 5/1032; A61B 5/1118; A61B 5/40; A61B 5/4893; A61B 18/20; A61B 90/20; A61B 2018/00577

(57) ABSTRACT

Methods of performing neural experiments on neuron-bearing living (NBL) organisms that include tracking movement of one or more NBL organisms via at least one first imaging objective and simultaneously collecting neural activity data via at least one second imaging objective. In some embodiments, the at least one first imaging objective is positioned along a first optical microscope light path and the at least one second imaging objective is positioned along a second optical microscope light path. In some embodiments, the first optical microscope light path is one of an inverted light path and an upright light path, and the second optical microscope light path is the other of the inverted light path and the upright light path. Various related methods, hardware, and software are also disclosed.

### RELATED APPLICATION DATA

The present application claims the benefit of priority to U.S. Provisional Patent Application Ser. No. 63/428,306, filed on Nov. 28, 2022, and titled “MICROSCOPE SYSTEMS, SOFTWARE, AND METHODS FOR PERFORMING NEURAL EXPERIMENTS ON LIVING AND/OR MOVING SUBJECTS”, which is incorporated by reference herein in its entirety.

### FIELD

The present disclosure generally relates to the field of microscopy of living tissue. In particular, the present disclosure is directed to microscope systems, software, and methods for performing neural experiments on living and/or moving subjects.

### BACKGROUND

Neural research uses a variety of living experimental subjects, including mice. The use of mice as living subjects has come under scrutiny as being inhumane to the mice, especially when the skull is opened for live-animal experiments involving neural stimulation and observation of any resulting behavioral response. Worms can provide living experimental subjects that are an alternative to mice. For example, microscopic C. elegans have a neural system comprising about 300 neurons, which is enough for some live-organism neural-stimulation experimentation. Importantly, C. elegans worms do not have high-functioning nervous systems like mice and, so, tend to not draw protestation for their use for live-organism neural-stimulation testing.

Unfortunately, limitations of conventional microscope systems constrain the neural-stimulation experiments that researchers can perform on C. elegans. For example, neural-stimulation experiments are typically performed by gluing the worms to a slide. As those skilled in the art will appreciate, gluing the worms to a slide is suboptimal for at least two reasons. First, the fixation can interfere with a researcher observing their stimulation-responsive behavior. Second, the fixation can interfere with a researcher selecting appropriate worms for study. Regarding the latter, sometimes a researcher wants to study only worms exhibiting a certain kind of behavior, such as the number of omega-bends that a worm makes in a specific time period. However, affixing the worms can interfere with the worms being able to exhibit the desired behavior.

### SUMMARY

In one implementation, the present disclosure is directed to a method of capturing data regarding a neuron-bearing living (NBL) organism using a microscope system. The method includes training a first imaging objective, having a first optical power along a first optical path, on at least a first portion of the NBL organism; training a second imaging objective on a neuron of the NBL organism, wherein the second imaging objective has a second optical path different from the first optical path and a second optical power higher than the first optical power; and while the first imaging objective is trained on the NBL organism and the second imaging objective is trained on the neuron, simultaneously: collecting activity-imaging data for the neuron using the second imaging objective; and collecting tracking data for the NBL organism using the first imaging objective.

In another implementation, the present disclosure is directed to a machine-readable storage medium containing machine-executable instructions for performing the method described above.

In yet another implementation, the present disclosure is directed to a microscope system, that includes hardware for performing the method described above; and the machine-readable storage medium described above for controlling the hardware to allow a user to perform the method.

### BRIEF DESCRIPTION OF THE DRAWINGS

For the purpose of illustration, the drawings show aspects of one or more embodiments of the disclosure. However, it should be understood that the present disclosure is not limited to the precise arrangements and instrumentalities shown in the drawings, wherein:

FIG. 1 is a partial elevational view/partial high-level block diagram illustrating an example microscope system made in accordance with aspects of the present disclosure;

FIG. 2A is an example extended mobile objective (EMO) assembly that can be used with the microscope system of FIG. 1, showing the imaging objective in a first translation position along an objective-translation axis and the EMO assembly pivoted to a first pivot position about a pivot axis;

FIG. 2B is an enlarged isometric view of the EMO assembly of FIG. 2A, showing the imaging objective in a second translation position different from the first translation position of FIG. 2A and the EMO assembly pivoted to a second pivot position different from the first pivot position of FIG. 2A;

FIG. 2C is an enlarged isometric view of the EMO assembly of FIGS. 2A and 2B, showing the EMO assembly in the same state shown in FIG. 2A but with a protective housing covering elements of the EMO assembly; and

FIG. 2D is an enlarged isometric view of the EMO assembly of FIGS. 2A and 2B, showing the EMO assembly in the same state shown in FIG. 2B but with the protective housing covering elements of the EMO assembly.

### DETAILED DESCRIPTION

### General Overview

In some aspects, the present disclosure is directed to microscope systems and methodologies for performing experiments, such as neural-stimulation experiments, on neuron-bearing living (NBL) organisms, such as, but not limited to, C. elegans nematodes and zebrafish larvae, among others. The present disclosure describes, among other things, unprecedented and transformative microscope systems that in some embodiments incorporate one or more of (i) a movable objective lens assembly that is the same as or similar to a movable objective lens assembly in accordance with U.S. Pat. No. 8,077,386 to Glaser et al., issued on Dec. 13, 2011, and titled “MOVABLE OBJECTIVE LENS ASSEMBLY FOR AN OPTICAL MICROSCOPE AND OPTICAL MICROSCOPES HAVING SUCH AN ASSEMBLY” (“the '386 patent”), (ii) technology for automatic, accurate, unbiased, high-throughput 3D cell quantification of fluorescently-labeled cells, (iii) a worm-tracking system that is the same as or similar to a worm-tracking system of U.S. Pat. No. 9,305,205 to Sprenger et al., issued on Apr. 5, 2016, and titled “METHODS AND SYSTEM FOR TRACKING MOVEMENT OF MICROSCOPIC WORMS AND WORM-LIKE ORGANISMS, AND SOFTWARE THEREFOR” (“the '205 patent”), and (iv) SCANIMAGE® software (available from MBF Bioscience, LLC, Williston, Vermont) or equivalent software that can control, for example, piezo objective positioners, shutters, and other components and aspects of a microscope system of the present disclosure. The above-referenced '386 and '205 patents are incorporated in this section in their entireties for the relevant teachings that skilled artisans can incorporate as desired into microscope systems, software, and methodologies disclosed herein.

Embodiments of the present disclosure enable researchers to, among other things, perform calcium imaging of one or more identified neurons in an NBL organism, such as, but not limited to a C. elegans worm within a group of freely moving and socially interacting worms on an agar plate (hereinafter “calcium imaging of individual neurons in a freely moving worm”), combined with advanced tracking and analysis of complex behavior of the worm and, as an option, simultaneous manipulation of the same and/or different identified neurons in the same worm using, for example, optogenetic stimulation or femtosecond laser ablation, respectively.

By providing this functionality, embodiments of the present disclosure enable researchers to perform unprecedented, next-generation studies into relationships between the function(s) of one or more identified neurons in a multi-neuron nervous system and its/their contribution(s) to the behavior of an animal in an environment that matches the studied NBL organism's cultivation conditions and with the optional ability to manipulate the function of the same and/or different identified neurons and study the consequences for the animal's behavior without need to change the animal's environment.

Embodiments of the present disclosure can perform, for example, identification of individual neurons in freely moving NBL organisms (e.g., worms) using a hybrid approach, supporting (i) the recently developed, multi-color, transgene NeuroPAL technology (that allows for nervous-system-wide neuronal identification in C. elegans) or similar technologies that might be developed in the future, and (ii) automatic detection of the position of densely distributed cell nuclei in 3D space. The Table immediately below provides nonlimiting examples of fluorescent dyes and genetically encoded calcium indicators that can be used with embodiments of the present disclosure.

TABLE

| | | | | | |
|---|---|---|---|---|---|
| | | Pan- | Supported by | Supported by | Channel |
| | | neuronal | Lumencor | Lumencor | needed for |
| Ex λ | Em λ | marker in | CELESTA | CELESTA | calcium |
| [nm] | [nm] | NeuroPAL | VBCTGRN | Quattro VCYR | imaging |

1) Fluorescent dyes used in the NeuroPAL technology

| | | | | | | |
|---|---|---|---|---|---|---|
| TagRFP-T | 555 | 584 | Yes | Yes (545 nm; 76% | No | No |
| | | | | of max. excitation) | | |
| TagBFP2 | 402 | 457 | No | Yes (405 nm; 94% | Yes (405 nm) | No |
| | | | | of max. excitation) | | |
| CyoFP1 | 497 | 589 | No | Yes (488 nm; 96% | Yes (488 nm) | Yes |
| | | | | of max. excitation) | | |
| mNeptune | 599 | 643 | No | Yes (577 nm; 70% | Yes (577 nm) | No |
| 2.5 | | | | of max. excitation) | | |

2) Fluorescent dyes used in for identifying cells in C. elegans

| | | | | | | |
|---|---|---|---|---|---|---|
| mCherry | 587 | 610 | — | Yes (577 nm; 88% | Yes (577 nm) | No |
| | | | | of max. excitation) | | |

3) GECIs used in C. elegans research

| | | | | | | |
|---|---|---|---|---|---|---|
| GCaMP6s | 497 | 512 | — | Yes (488 nm; 96% | Yes (488 nm; 96% | Yes |
| | | | | of max. excitation) | of max. excitation) | |
| YC3.60 | | | | | | |
| Ribo- | | | | | | |
| GCaMP | | | | | | |

Source: https://www.fpbase.org/

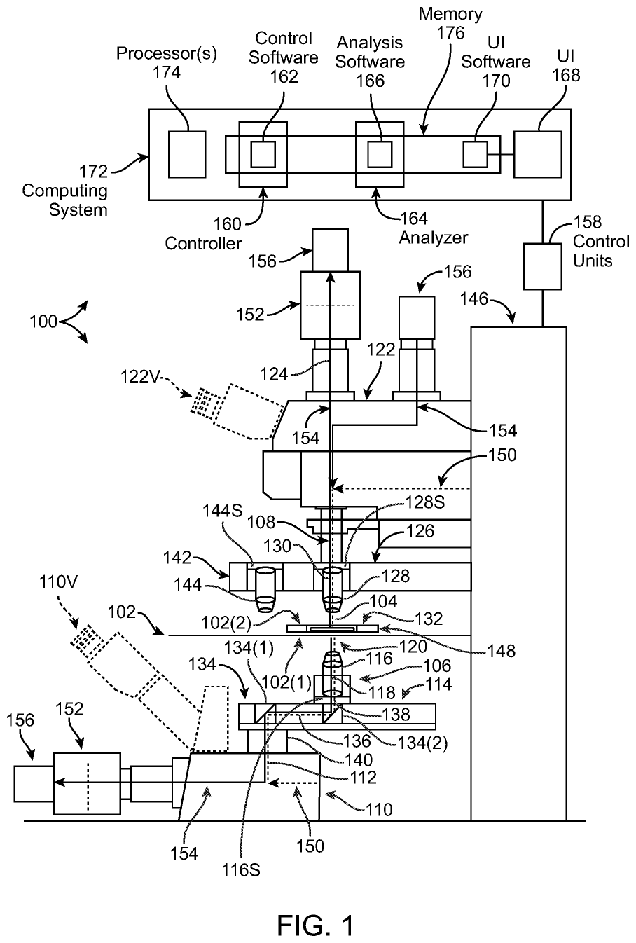

Higher-end, i.e., more feature-laden, embodiments of microscope systems of the present disclosure, whose general functioning principles are illustrated in the example of FIG. 1, can, for example, allow investigators to perform experiments as follows: (1) automatically identify (using a low-power objective (“O1”) in a first optical path and reflected light coming from a side ring illuminator) a certain worm (or other NBL organism) within a group of freely moving worms based on a specific, pre-defined behavior (e.g., a worm that shows the highest number of omega bends in a certain period of time) or any worm of interest, (2) automatically identify (using a high-power objective (“O2”) in a second optical path that is parallel to the first optical path, and four lasers with different wavelengths) individual neurons in the freely moving worm, (3) simultaneously perform calcium imaging of one or more identified neurons and advanced tracking and analysis of complex behavior of the worm, (4) automatically switch the first optical path to a high-power, fluorescence objective (“O3”) using an objective changer, (5) automatically navigate the high-power objective O3 to the same and/or different, identified neurons in the same worm, and perform optogenetic stimulation or femtosecond laser ablation of these neurons, (6) automatically switch the first optical path back to the low-power objective O1, and continue to simultaneously perform calcium imaging of one or more identified neurons and advanced tracking and analysis of complex behavior of the worm, and (7) repeat the immediately preceding functions 4 to 6 with optogenetic stimulation or femtosecond laser ablation of different neurons according to the study's protocol.

Embodiments of the present disclosure may have differing numbers of features. For example, some standard, i.e., less feature laden, embodiments of the present disclosure can enable investigators to perform, for example, the above functions 1 to 3 but not the above functions 4 to 6. Other functions and combinations of functions are possible. Embodiments of the present disclosure enable researchers to also/alternatively investigate NBL organisms other than C. elegans nematodes, including zebrafish larvae, among others.

### Example Microscope System

Before proceeding with describing an example microscope system and components thereof, it is noted that the numerical identifiers such as “first”, “second”, “third”, etc., used throughout this disclosure and in the appended claims are used for convenience to simply distinguish differing ones of same or similar elements or features from one another when multiple ones of such elements or features are present in any corresponding embodiment or portion(s) thereof being described or claimed. Consequently, these numerical identifiers are not to be interpreted as denoting any sort of ordering, ranking, preference, etc., of such elements or features.

Referring now to the drawings, FIG. 1 illustrates an example microscope system 100 made in accordance with aspects of the present disclosure. In this example, the microscope system 100 includes a movable stage 102 that supports a specimen 104, which can be any suitable specimen meeting requirements discussed above. The movable stage 102 may be a motorized XY specimen stage having a first side 102(1) and a second side 102(2) spaced from the first side so that the specimen 104 is viewable from each of the first and second sides by, respectively, a first optical microscope light path 106 and a second optical microscope light path 108.

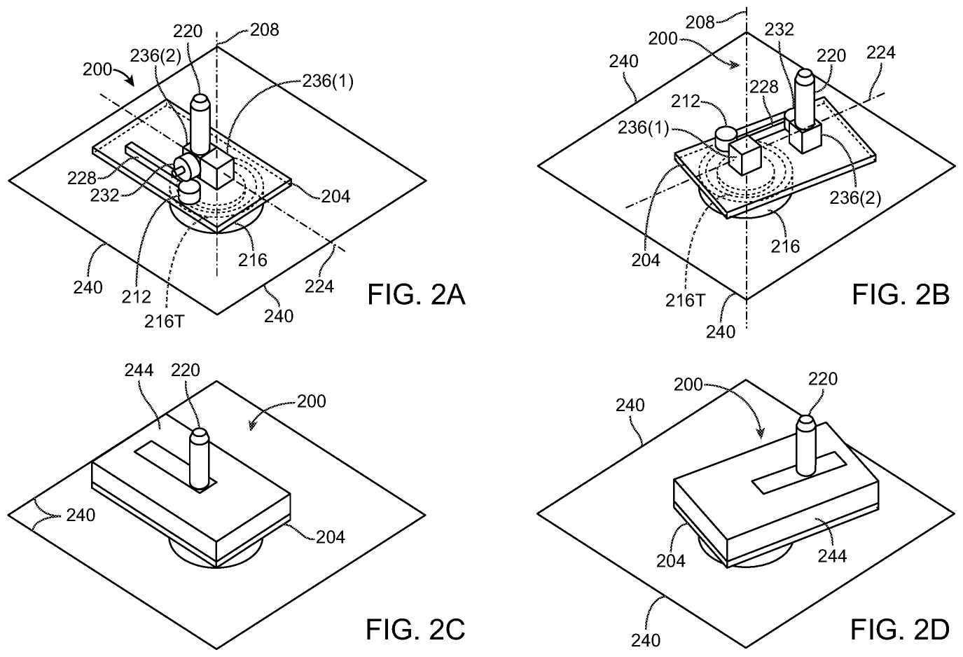

In the embodiment shown, the first optical microscope light path 106 is provided by an inverted fluorescence microscope that comprises a first imaging body 110 having a first primary optical axis 112 and including a first movable objective assembly 114. The first movable objective assembly 114 includes a first imaging objective 116 located for viewing the specimen 104 from the first side 102(1) of the movable stage 102 when the specimen is supported by the movable stage. The first imaging objective 116 may be supported by a piezo objective scanner 116S (not shown in the example of FIGS. 2A through 2D) and has a first optical axis 118 and a first field of view 120, and the first movable objective assembly 114 is configured to move the first imaging objective in a direction parallel to the first optical axis and in a direction perpendicular to the first optical axis, ideally over a region that allows the first imaging objective to traverse the entire specimen 104. In some embodiments, the first imaging objective 116 has a relatively high power (e.g., 40× or 60×, among others). In some embodiments, a first optical viewer 110V may optionally be operatively engaged with the first imaging body 110 to allow a user to observe the first field of view 120.

In this example, the second optical microscope light path 108 is provided by an upright fluorescent microscope that comprises a second imaging body 122 having a second primary optical axis 124. A second movable objective assembly 126 is operatively located relative to the second imaging body 122 and includes a second imaging objective 128 located for viewing the specimen 104 from the second side 102(2) of the movable stage 102 when the specimen is supported by the movable stage. The second imaging objective 128 has a second optical axis 130 and a second field of view 132, and the second movable objective assembly 126 is configured to move the second imaging objective in a direction parallel to the second optical axis. In some embodiments, a second optical viewer 122V may optionally be operatively engaged with the second imaging body 122 to allow a user to observe the second field of view 132. In some embodiments, when one or both of the first and second optical viewers 110V and 122V are not present, both of the first and second optical microscope light paths 106 and 108 may be considered to be provided by a single optical microscope having both inverted and upright portions.

The first movable objective assembly 114 comprises a mirror system 134 that includes a plurality of mirrors, here a first mirror 134(1) and a second mirror 134(2). In this example, the first mirror 134(1) is fixed along the first primary optical axis 112, is pivotable thereabout, and directs light between the first primary optical axis and a third optical axis 136 perpendicular to the first primary optical axis. Also in this example, the second mirror 134(2) is fixed relative to the first imaging objective 116 and directs light between the third optical axis 136 and a fourth optical axis 138 that is parallel to the first optical axis 118 of the first imaging objective 116. In the embodiment shown, the first movable objective assembly 114 is supported on the first imaging body 110 via a first support 140 that may include a Z-axis focus motor (not shown) and/or allow the mirror system 134 to pivot about the first primary optical axis 112. In the embodiment shown, the first movable objective assembly 114 may be referred to as an extended mobile objective (EMO) assembly, with an example EMO assembly 200 illustrated in FIGS. 2A through 2D.

As seen in FIGS. 2A and 2B, in this example, the EMO assembly 200 includes a pivotable support 204 that is pivoted about a pivot axis 208 by a suitable pivot motor 212, such as, for example, a stepper motor, that operatively engages a track 216T of a fixed base 216. An imaging objective 220, which corresponds to the first imaging objective 116 of FIG. 1, is translationally mounted to the pivotable support 204 so as to be movable along an objective-translation axis 224, for example, via suitable translation mechanism, such as the rack 228 and pinion 232 mechanism illustrated, among others. In this example, the EMO assembly 200 includes first and second mirrors 236(1) and 236(2) that correspond, respectively, to the first and second mirrors 134(1) and 134(2) of FIG. 1 and provide the same functions as those mirrors of FIG. 1. The first mirror 236(1) is fixed to the pivotable support 204, while the second mirror 236(2) supports the imaging objective 220 and is moved therewith by the translation mechanism, here, the rack 228 and pinion 232 mechanism. As is evident from the fixed reference grid 240, FIG. 2A shows the imaging objective 220 in a first translation position along the objective-translation axis 224 and the pivotable support 204 pivoted to a first pivot position about the pivot axis 208, and FIG. 2B shows the imaging objective in a second translation position along the objective-translation axis that is different from the first translation position of FIG. 2A and the pivotable support pivoted to a second pivot position about the pivot axis that is different from the first pivot position of FIG. 2A. FIGS. 2C and 2D correspond, respectively, to FIGS. 2A and 2B but show components of the EMO assembly 200 contained in a protective housing 244.

It is noted that the example EMO assembly 200 is shown here in non-scaled manner. In some embodiments, linear movement of the imaging objective 220 may, for example, be limited to a distance of 5 cm. The housing provides a light-proof box having a flexible opening and covers the mirrors, motors, and other components of the EMO assembly 200. By means of the Z-axis focus motor mentioned above relative to FIG. 1, the EMO assembly 200, and the piezo objective scanner 116S (FIG. 1), when the EMO assembly is included in the microscope system 100 of FIG. 1, the imaging objective 220 can be moved relative to the specimen 104 independently in orthogonal directions X, Y and Z. Further details of EMO assemblies that can be used for the EMO assembly 200 of FIGS. 2A through 2D and the first movable objective assembly 114 can be found in the '386 patent incorporated by reference above.

Referring again to FIG. 1, in the embodiment shown the second movable objective assembly 126 comprises a motorized objective changer 142 that enables moving the second imaging objective 128 into and out of the second primary optical axis 124 and to swap one or more third imaging objectives 144 into and out of the second primary optical axis. For example, the motorized objective changer 142 can allow the second optical microscope 108 to operate with a relatively low-power imaging objective 128 for advanced tracking and analysis of complex behavior of worms or other NBL organism in the specimen 104 and allow the second optical microscope to operate with a relatively high-power imaging objective 144 for calcium imaging and/or manipulation of one or more neurons in a given worm or NBL organism using, for example, optogenetics or femtosecond laser ablation. In some embodiments, the second and third imaging objectives 128 and 144 of the second optical microscope 108 are mounted on piezo objective scanners 128S and 144S, respectively. During tracking and analysis of behavior, the worms or NBL organisms in the specimen 104 are illuminated using a side-ring illuminator 148. Calcium imaging using the second optical microscope 108 and manipulation of neurons using optogenetics or femtosecond laser ablation can be performed with separate lasers (not shown in FIG. 1, but see excitation light 150 discussed below).

In some embodiments, the microscope system 100 may further comprise, among other things customarily present in microscope systems of the general sort at issue in the microscope system 100, any one or more, including all, of the following:
a motorized Z-column 146 that moves the second imaging body 122 in a direction parallel to the second primary optical axis 124 to enable, for example, swapping the second and third imaging objectives 128 and 144, respectively, by means of the objective changer 142 without damaging the second and/or third imaging objective, the movable stage 102, the specimen 104, and/or the second imaging body 122;
a plurality of lasers (not shown) whose excitation light 150 illuminates the specimen 104 on the first and second side 102(1) and 102(2) of the movable stage 102;
a plurality of spinning-disk confocal microscopy units 152 (e.g., X-Light V2 units available from CrestOptics, Rome, Italy) that generate confocal imaging based on the emission light 154 that is emitted from the specimen 104 after illumination with the excitation light 150;
one or more cameras 156 (e.g., as available from Hamamatsu Corporation; Hamamatsu City, Japan) for collecting the emission light 154 emitted by the specimen 104 (in some embodiments, camera 156(3) may be provided to capture light coming from the side-ring illuminator 148);
control units (collectively represented at 158 for simplicity) for operating, respectively, the first and second imaging bodies 110 and 122, the first and second optical viewers 110V and 122V (when present), the first and second movable objective assemblies 114 and 126, the side ring illuminator 148, the lasers (not shown), the first and second imaging bodies 110 and 122, and each cameras 156;
a controller 160 in operative communication with the control units 158;
control software 162 aboard the controller 160 to control the plurality of control units 158 for operating, respectively, the first and second imaging bodies 110 and 122, the first and second optical viewers 110V and 122V (when present), the first and second movable objective assemblies 114 and 126, the side ring illuminator 148, the lasers (not shown), the first and second imaging bodies 110 and 122, and each cameras 156;
an analyzer 164 that receives output of each camera 156;
analysis software 166 aboard the analyzer 164 to analyze the output of each camera 156;
a user interface (UI) 168 in operative communication with the analyzer and that includes input and output devices (not shown) needed to utilize the microscope system 100, including, but not limited to, one or more display devices for displaying images from cach camera, data that the analysis software generates, and UI screens that allow a user to control operations of the microscope system 100 and the analyzer, and one or more user-input devices for allowing the user to exert such control;
UI software 170 for providing and controlling the UI; and
a computing system 172 that contains the controller 160, the analyzer 164, the UI 168 and the UI software 170, and includes one or more processors 174 (e.g., general processing unit(s), field programmable gate array(s), processor(s) aboard one or more application specific integrated circuit, or any other suitable microprocessor) and suitable memory (176).

As those skilled in the art will readily understand, the above descriptions of the computing hardware, software, and associated UI(s) and machine interfaces for interfacing with users and the various hardware components of the microscope system 100 and related hardware are to be understood as being general in nature and should not be taken as describing any particular types and arrangements of computing hardware. For example, the terms “analyzer”, “controller”, and any like term, while they can describe physically discrete components, more typically describe a particular functionality that is provided by any suitable combination of hardware and software. For example, the controller may, in fact, be implemented as any suitable type of control system, such as a centralized control system, a distributed control system, or a hybrid of centralized and distributed control systems.

In some embodiments, the optical microscope system 100 may further include particular software, such as the worm-tracking software of the '205 patent incorporated by reference above, and/or the SCANIMAGE® software mentioned above, or any suitable variation thereof. As those skilled in the art will readily appreciate, in the context of the functional components noted above, such as the controller 160, control software 162, analyzer 164, analyzer software 166, UI 168, and UI software 170, such particular software can be part of and/or can interact with one or more of these components to provide the requisite functionality(ies).

Any software specifically noted herein and/or needed to make the example optical microscope system 100, or any other optical microscope system or setup disclosed herein or obvious to someone skilled in the art without undue experimentation may be provided and/or present in any suitable machine-readable storage medium (media) (e.g., the memory 176) compatible with the provided hardware (e.g., the processor(s) 174). It is noted that the term “machine-readable storage medium” includes any one or more types of hardware memory, including, but not limited to, any type(s) of volatile and/or nonvolatile hardware memory (e.g., RAM, ROM, optical, magnetic, bubble, etc.) to the exclusion of transitory signals, such as signals present on a carrier wave or in a pulsed transmission, such as an optical transmission or an acoustic transmission. Those skilled in the art will readily understand the wide variety of computing hardware that can be used such that further descriptions are not necessary herein for those skilled in the art to be able to practice the inventions of the present disclosure to their fullest scope based on the descriptions provided herein and in the references incorporated by reference herein.

In a first additional example optical microscope setup, the optical microscope setup includes:
a movable stage for supporting a specimen, having a first side and a second side spaced from the first side, so that each of the first and second sides is viewable;
a first optical microscope light path comprising:
   a first imaging body having a first primary optical axis; and
   a first movable objective assembly having a first objective located for viewing the first side of the specimen when the specimen is supported by the movable stage, the first objective having a first optical axis and a first field of view, and the first movable objective assembly configured to move the first objective in a direction parallel to the first optical axis and to move the first objective in a direction perpendicular to the first optical axis; and
a second optical microscope light path comprising:
   a second imaging body having a second primary optical axis; and
   second movable objective assembly having a second objective located for viewing the second side of the specimen when the specimen is supported by the movable stage, the second objective having a second optical axis and a second field of view, and the second movable objective assembly configured to move the second objective in a direction parallel to the second optical axis;
wherein the first movable objective assembly comprises a mirror system that includes:
   plurality of mirrors, including:
   first mirror fixed along the first primary optical axis and pivotable thereabout, the first mirror for directing light from the first primary optical axis to a third optical axis perpendicular to the first optical axis to along a fourth optical axis perpendicular to the third optical axis; and
   second mirror fixed relative to the first objective and movable in a direction parallel to the third optical axis, the second mirror for directing light from the third optical axis to the fourth optical axis perpendicular to the third optical axis to along the first field of view; and
   first support supporting the first plurality of mirrors so as to allow the first plurality of mirrors to pivot about the first primary optical axis.

The following embodiments involved determining which microscope light path includes an inverted imaging body and which one includes an upright imaging body:

In some embodiments of the first additional example optical microscope setup, the first imaging body is an inverted imaging body and the second imaging body is an upright imaging body.

In some embodiments of the first additional example optical microscope setup, the first imaging body is an upright imaging body and the second imaging body is an inverted imaging body.

In some embodiments of the first additional example optical microscope setup, the first and second imaging bodies are embodied in first and second optical microscopes.

In some embodiments of the first additional example microscope setup, the first and second imaging bodies are embodied in a single optical microscope.

The following embodiments involve differing specimens (C. elegans worms, zebrafish larvae, and others):

In some embodiments of the first additional example optical microscope setup, the specimen contains an agar plate in a transparent container.

In some embodiments of the first additional example optical microscope setup, the specimen contains one or more animals that freely move and socially interact on the agar plate.

In some embodiments of the first additional example optical microscope setup, the one or more animals are Caenorhabditis elegans nematodes.

In some embodiments of the first additional example optical microscope setup, the one or more animals are other nematodes than Caenorhabditis elegans.

In some embodiments of the first additional example optical microscope setup, the one or more animals are insects.

In some embodiments of the first additional example optical microscope setup, the one or more animals are amphibians.

In some embodiments of the first additional example optical microscope setup, the one or more animals are mammals.

In some embodiments of the first additional example optical microscope setup, the specimen contains a liquid in a transparent container.

In some embodiments of the first additional example optical microscope setup, the specimen contains one or more animals that freely move and socially interact in the liquid.

In some embodiments of the first additional example optical microscope setup, the one or more animals are Caenorhabditis elegans nematodes.

In some embodiments of the first additional example optical microscope setup, the one or more animals are other nematodes than Caenorhabditis elegans.

In some embodiments of the first additional example optical microscope setup, the one or more animals are zebrafish larvae.

In some embodiments of the first additional example optical microscope setup, the one or more animals are other larvae than zebrafish larvae.

In some embodiments of the first additional example optical microscope setup, the one or more animals are fishes.

In some embodiments of the first additional example optical microscope setup, the one or more animals are insects.

In some embodiments of the first additional example optical microscope setup, the one or more animals are amphibians.

In some embodiments of the first additional example optical microscope setup, the one or more animals are mammals.

The following embodiments are directed to an objective changer of an inverted microscope:

In some embodiments of the first additional example optical microscope setup, the first movable objective assembly comprises a first objective changer that enables to move the first objective into and out of the first optical axis and one or more third objectives into and out of the first optical axis.

In some embodiments of the first additional example optical microscope setup, the first and third objectives are moved in a linear motion in a direction perpendicular to the first optical axis into and out of the first optical axis.

In some embodiments of the first additional example optical microscope setup, the first and third objectives are moved in a swinging motion around an axis that is perpendicular to the first optical axis into and out of the first optical axis.

In some embodiments of the first additional example optical microscope setup, the optical microscope setup includes a motorized Z-column that moves the movable stage, the specimen and the second imaging body in a direction parallel to the second primary optical axis to enable to change the first and third objective by means of the first objective changer without damaging the first and third objective, the movable stage, the specimen and the first imaging body.

The following embodiments are directed to an objective changer of an upright microscope:

In some embodiments of the first additional example optical microscope setup, the second movable objective assembly comprises a second objective changer that enables to move the second objective into and out of the second primary optical axis and one or more fourth objectives into and out of the second primary optical axis.

In some embodiments of the first additional example optical microscope setup, the second and fourth objectives are moved in a linear motion in a direction perpendicular to the second primary optical axis into and out of the second primary optical axis.

In some embodiments of the first additional example optical microscope setup, the second and fourth objectives are moved in a swinging motion around an axis that is perpendicular to the second primary optical axis into and out of the second primary optical axis.

In some embodiments of the first additional example optical microscope setup, the optical microscope setup includes a motorized Z-column that moves the second imaging body in a direction parallel to the second primary optical axis to enable to change the second and fourth objective by means of the second objective changer without damaging the first and fourth objective, the movable stage, the specimen and the second imaging body.

Embodiments directed to an objective changer for both inverted and upright microscopes:

In some embodiments of the first additional example optical microscope setup:
the first movable objective assembly comprises a first objective changer that enables to move the first objective into and out of the first optical axis and one or more third objectives into and out of the first optical axis, and
the second movable objective assembly comprises a second objective changer that enables to move the second objective into and out of the second primary optical axis and one or more fourth objectives into and out of the second primary optical axis.

In some embodiments of the first additional example optical microscope setup:
the first and third objectives are moved in a linear motion in a direction perpendicular to the first optical axis into and out of the first optical axis, and
the second and fourth objectives are moved in a linear motion in a direction perpendicular to the second primary optical axis into and out of the second primary optical axis.

In some embodiments of the first additional example optical microscope setup:
the first and third objectives are moved in a linear motion in a direction perpendicular to the first optical axis into and out of the first optical axis, and
the second and fourth objectives are moved in a swinging motion around an axis that is perpendicular to the second primary optical axis into and out of the second primary optical axis.

In some embodiments of the first additional example optical microscope setup:
the first and third objectives are moved in a swinging motion around an axis that is perpendicular to the first optical axis into and out of the first optical axis, and
the second and fourth objectives are moved in a linear motion in a direction perpendicular to the second primary optical axis into and out of the second primary optical axis.

In some embodiments of the first additional example optical microscope setup:
the first and third objectives are moved in a swinging motion around an axis that is perpendicular to the first optical axis into and out of the first optical axis, and
the second and fourth objectives are moved in a swinging motion around an axis that is perpendicular to the second primary optical axis into and out of the second primary optical axis.

In some embodiments of the first additional example optical microscope setup, the optical microscope setup includes a motorized Z-column that moves the second imaging body in a direction parallel to the second primary optical axis to enable to change the first, second, third and fourth objectives by means of the first and second objective changers without damaging the first, second, third and fourth objectives, the movable stage, the specimen and the first and second imaging bodies.

Embodiments that use a radial scanning objective (RSO) assembly instead of an EMO assembly:

In some embodiments of the first additional example optical microscope setup, the third optical axis and the fourth optical axis are coaxial with one another.

In some embodiments of the first additional example optical microscope setup, the mirror system consists essentially of four mirrors such that the mirror system includes a third mirror and a fourth mirror working cooperatively with one another so as to fold the fourth optical path along the third optical path.

In some embodiments of the first additional example optical microscope setup, the fourth mirror is fixed relative to the first mirror and the second and third mirrors are movable along the fourth optical path.

In some embodiments of the first additional example optical microscope setup, the first and second primary optical axes are substantially parallel to one another and spaced from one another.

In some embodiments of the first additional example optical microscope setup, the first and second primary optical axes are substantially parallel to one another and not spaced from one another.

Embodiments having additional technical details of the first microscope setup:

In some embodiments of the first additional example optical microscope setup, the first optical microscope is configured so that the first or third objective is movable only in a circular sector having a central angle less than 180°.

In some embodiments of the first additional example optical microscope setup, the first, second, third and fourth objectives have intentionally differing magnifying powers.

In some embodiments of the first additional example optical microscope setup, the first, second, third and fourth objectives have intentionally the same magnifying power.

In some embodiments of the first additional example optical microscope setup, one or more than one of the second and fourth objectives are relatively low power scouting objectives for use in identifying within the specimen a feature of interest, and one or more than one of the first and third objectives are relatively high-power detail objectives for imaging the feature of interest at a higher power.

In some embodiments of the first additional example optical microscope setup, one or more than one of the first and third objectives are relatively low power scouting objectives for use in identifying within the specimen a feature of interest, and one or more than one of the second and fourth objectives are relatively high-power detail objectives for imaging the feature of interest at a higher power.

In some embodiments of the first additional example optical microscope setup, the optical microscope setup includes a controller programmed to move the first or third objective to the feature of interest in response to identification of the feature of interest using the second or fourth objective.

Additional microscope setups, wherein the first microscope is an inverted microscope and the second microscope is an upright microscope:

In some embodiments of the first additional example optical microscope setup, the optical microscope setup includes a controller programmed to move the second or fourth objective to the feature of interest in response to identification of the feature of interest using the first or third objective.

In a second additional example optical microscope setup, the optical microscope setup includes:
a movable stage for supporting a specimen, having a first side and a second side spaced from the first side, so that each of the first and second sides is viewable;
a first optical microscope light path comprising:
   first imaging body having a first primary optical axis; and
   first movable objective assembly having a first objective located for viewing the first side of the specimen when the specimen is supported by the movable stage, the first objective having a first optical axis and a first field of view, and the first movable objective assembly configured to move the first objective in a direction parallel to the first optical axis and to move the first objective in a direction perpendicular to the first optical axis; and
a second optical microscope light path comprising:
   second imaging body having a second primary optical axis; and
   second movable objective assembly having a second objective located for viewing the second side of the specimen when the specimen is supported by the movable stage, the second objective having a second optical axis and a second field of view, and the second movable objective assembly configured to move the second objective in a direction parallel to the second optical axis;
wherein:
   first and second primary optical axes are substantially parallel to one another and spaced from one another; and
   first microscope is configured so that the first objective is movable only in a circular sector having a central angle less than 180°, the circular sector extending generally away from the first primary optical axis.

In some embodiments of the second additional example optical microscope setup, the first movable objective assembly comprises a first mirror system that includes:
a first plurality of mirrors, including:
   a first mirror fixed along the first primary optical axis and pivotable thereabout, the first mirror for directing light from a third optical axis perpendicular to the first optical axis to along the first primary optical axis; and
   second mirror fixed relative to the first objective and movable in a direction parallel to the third optical axis, the second mirror for directing light from the first field of view along a fourth optical axis parallel to the third optical axis; and
a first support supporting the first plurality of mirrors so as to allow the first plurality of mirrors to pivot about the first primary optical axis.

In some embodiments of the second additional example optical microscope setup, the third and fourth optical axes are coaxial with one another.

In some embodiments of the second additional example optical microscope setup, the first mirror system consists essentially of four mirrors such that the first mirror system includes a third mirror and a fourth mirror working cooperatively with one another so as to fold the fourth optical path along the third optical path.

In some embodiments of the second additional example optical microscope setup, the fourth mirror is fixed relative to the first mirror and the second and third mirrors are movable along the fourth optical path.

In some embodiments of the second additional example optical microscope setup, the first optical microscope light path exists in an inverted microscope and the second optical microscope light path exists in an upright microscope.

In some embodiments of the second additional example optical microscope setup, the first optical microscope light path exists in an upright microscope and the second optical microscope light path exists in an inverted microscope.

In some embodiments of the second additional example optical microscope setup, the first and second imaging bodies are embodied in first and second optical microscopes.

In some embodiments of the second additional example microscope setup, the first and second imaging bodies are embodied in a single optical microscope.

In some embodiments of the second additional example optical microscope setup, the first and second objectives have intentionally differing magnifying powers.

In some embodiments of the second additional example optical microscope setup, the first and second objectives have intentionally identical magnifying powers.

In some embodiments of the second additional example optical microscope setup, the second objective is a relatively low power scouting lens for use in identifying within the specimen a feature of interest, and the first objective is a relatively high-power detail lens for imaging the feature of interest at a higher power.

In some embodiments of the second additional example optical microscope setup, the first objective is a relatively low power scouting lens for use in identifying within the specimen a feature of interest, and the second objective is a relatively high-power detail lens for imaging the feature of interest at a higher power.

In some embodiments of the second additional example optical microscope setup, the optical microscope setup further includes a controller programmed to move the first objective to the feature of interest in response to identification of the feature of interest using the second objective lens.

In some embodiments of the second additional example optical microscope setup, the optical microscope setup further includes a controller programmed to move the second objective to the feature of interest in response to identification of the feature of interest using the first objective lens.

In a third additional example optical microscope setup, the optical microscope setup includes:

hardware for performing any one of the methods discussed in the section immediately below titled “Example Methods and Corresponding Software”; and

a machine-readable storage medium for controlling the hardware to allow a user to perform the method, wherein the machine-readable storage medium is the machine-readable storage medium discussed in the section immediately below titled “Example Methods and Corresponding Software”.

### Example Methods and Corresponding Software

With references to relevant features and functionalities of microscope systems of the present disclosure described above, any suitably configured one of such microscope systems may be used to perform any one or more of a variety of methods. Examples of these methods are provided below.

In a first example, a method of capturing data regarding an NBL organism is performed using a microscope system of the present disclosure. In this example, the method includes:
training a first imaging objective, having a first optical power along a first optical path, on at least a first portion of the NBL organism;
training a second imaging objective on a neuron of the NBL organism, wherein the second imaging objective has a second optical path different from the first optical path and a second optical power higher than the first optical power; and
while the first imaging objective is trained on the NBL organism and the second imaging objective is trained on the neuron, simultaneously:
   activity-imaging data for the neuron using the second imaging objective; and
   collecting tracking data for the NBL organism using the first imaging objective.

In some embodiments of the first example method, the method may further include recording the activity-imaging data and the tracking data so that the activity-imaging data and the tracking data is or can be synced with one another for playback.

In some embodiments of the first example method, the activity-imaging data is generated using a calcium indicator.

In some embodiments of the first example method, the activity-imaging data is generated using a voltage indicator.

In some embodiments of the first example method, the activity-imaging data is generated using a biosensor for a neurotransmitter.

In some embodiments of the first example method, the activity-imaging data is generated using a biosensor for a neuromodulator.

In some embodiments of the first example method:
the first and second imaging objectives have corresponding first and second fields of view;
the NBL organism is moving while performing the method; and
training the second imaging objective includes moving the second field of view so as to keep the neuron in the second field of view.

In some embodiments of the first example method, moving the second field of view includes moving a stage that supports the NBL organism.

In some embodiments of the first example method, moving the second field of view includes moving the second imaging objective.

In some embodiments of the first example method, training the first imaging objective includes moving the first field of view so as to keep at least a second portion of the NBL organism in the first field of view, wherein the second portion is the same as or different from the first portion.

In some embodiments of the first example method, moving the first field of view includes moving a stage that supports the NBL organism.

In some embodiments of the first example method, moving the first field of view includes moving the first imaging objective.

In some embodiments of the first example method, the neuron is a desired neuron, and the method further includes identifying the desired neuron using a neuron-identification scheme.

In some embodiments of the first example method, the neuron-identification scheme is a color-based scheme.

In some embodiments of the first example method, the color-based scheme is based on transgenic expression of one or more fluorophores.

In some embodiments of the first example method, the method further includes executing a neuron-locating algorithm to estimate a location of the neuron within the NBL organism based on imaging using the first imaging objective.

In some embodiments of the first example method, the method further includes automatically moving the second field of view based on the location estimated.

In some embodiments of the first example method, the method further includes performing a response-provoking operation on the neuron, one or more other neurons, or both the neuron and the one or more other neurons, and either 1) performing the simultaneous collecting of both the activity-imaging data and the tracking data or 2) continuing to collect both the activity-imaging data and the tracking data.

In some embodiments of the first example method, the response-provoking operation comprises optogenetic stimulation.

In some embodiments of the first example method, the response-provoking operation comprises laser ablation.

In some embodiments of the first example method, performing the response-provoking operation uses a third imaging objective.

In some embodiments of the first example method, the method further includes:
switching—in the third imaging objective for the first imaging objective along the first optical path to perform the response-provoking operation; and
after performing the response-provoking operation, switching-back the first imaging objective for the third imaging objective along the first optical path.

In some embodiments of the first example method, the method further includes selecting the NBL organism out of a plurality of like NBL organisms based on at least one criterion.

In some embodiments of the first example method, the at least one criterion comprises a manner of movement.

In some embodiments of the first example method, the method further includes automatically analyzing behavior of the NBL organism based on the tracking data collected via the first imaging objective.

In some embodiments of the first example method, the NBL organism is a C. elegans nematode.

In some embodiments of the first example method, the NBL organism is a zebrafish larvae.

In some embodiments of the first example method, the NBL organism is supported by a stage and the first and second imaging objectives are located on opposite sides of the stage.

In some embodiments of the first example method, the first and second optical axes form an angle of 180° with one another.

In some embodiments of the first example method, the first and second optical axes form an angle with one another that is greater than 0° and less than 180º.

In some embodiments of the first example method, the first and second optical axes form an angle with one another that is greater than 0° and less than 90°.

In a second example, a method of performing microscopy on a specimen is performed using an optical microscope setup that includes a first objective having a first magnification power and first field of view and a second objective having a second magnification power smaller than the first magnification power and a second field of view, wherein the specimen has a first side and a second side spaced from the first side and the first objective is located on the first side of the specimen and the second objective is located on the second side of the specimen. In this example, the method includes:
determining a location of a feature of interest based on a first position of the second objective relative to the specimen; and
based on the determining of the location of the feature of interest, automatedly moving the first objective to a second position so that the first field of view contains at least a portion of the feature of interest.

In some embodiments of the second example method, the optical microscope setup further includes:
a first imaging body; and
a second imaging body fixed relative to the first imaging body;
wherein the first objective is movable relative to the first imaging body and the second objective is movable relative to the second imaging body;

and the second example method includes prior to the determining of the location of the feature, moving the first objective to the first position by moving the first objective relative to the first imaging body; wherein the moving of the specimen relative to the second objective to the second position includes moving the specimen relative to the second imaging body.

In some embodiments of the second example method:
the first imaging body has a first primary optical axis;
the second imaging body has a second primary optical axis;
the first objective has a third optical axis;
the second objective has a fourth optical axis; and
the moving of the first objective relative to the first imaging body includes, when the third optical axis is parallel to, and spaced from, the first primary optical axis, pivoting the first objective about the first primary optical axis.

In some embodiments, any one of the above example methods is embodied in machine-executable instructions stored in a machine-readable storage medium.

Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve aspects of the present disclosure. Accordingly, this description is meant to be taken only by way of example, and not to otherwise limit the scope of this invention.

Exemplary embodiments have been disclosed above and illustrated in the accompanying drawings. It will be understood by those skilled in the art that various changes, omissions and additions may be made to that which is specifically disclosed herein without departing from the spirit and scope of the present invention.

What is claimed is:

1. A method of capturing data regarding a neuron-bearing living (NBL) organism using a microscope system, the method comprising:
training a first imaging objective, having a first optical power along a first optical path, on at least a first portion of the NBL organism;
training a second imaging objective on a neuron of the NBL organism, wherein the second imaging objective has a second optical path different from the first optical path and a second optical power higher than the first optical power; and
while the first imaging objective is trained on the NBL organism and the second imaging objective is trained on the neuron, simultaneously:
collecting activity-imaging data for the neuron using the second imaging objective; and
collecting tracking data for the NBL organism using the first imaging objective.

2. The method of claim 1, further comprising recording the activity-imaging data and the tracking data so that the activity-imaging data and the tracking data is or can be synced with one another for playback.

3. The method of claim 1, wherein:
the first and second imaging objectives have corresponding first and second fields of view;
the NBL organism is moving while performing the method; and
training the second imaging objective includes moving the second field of view so as to keep the neuron in the second field of view.

4. The method of claim 3, wherein moving the second field of view includes moving a stage that supports the NBL organism.

5. The method of claim 3, wherein moving the second field of view includes moving the second imaging objective.

6. The method of claim 3, wherein training the first imaging objective includes moving the first field of view so as to keep at least a second portion of the NBL organism in the first field of view, wherein the second portion is the same as or different from the first portion.

7. The method of claim 6, wherein moving the first field of view includes moving a stage that supports the NBL organism.

8. The method of claim 6, wherein moving the first field of view includes moving the first imaging objective.

9. The method of claim 1, wherein the neuron is a desired neuron, and the method further includes identifying the desired neuron using a neuron-identification scheme.

10. The method of claim 9, wherein the neuron-identification scheme is a color-based scheme.

11. The method of claim 10, wherein the color-based scheme is based on transgenic expression of one or more fluorophores.

12. The method of claim 9, further comprising executing a neuron-locating algorithm to estimate a location of the neuron within the NBL organism based on imaging using the first imaging objective.

13. The method of claim 12, further comprising automatically moving the second field of view based on the location estimated.

14. The method of claim 1, further comprising performing a response-provoking operation on the neuron, one or more other neurons, or both the neuron and the one or more other neurons, and either 1) performing the simultaneous collecting of both the activity-imaging data and the tracking data or 2) continuing to collect both the activity-imaging data and the tracking data.

15. The method of claim 14, wherein the response-provoking operation comprises optogenetic stimulation.

16. The method of claim 14, wherein the response-provoking operation comprises laser ablation.

17. The method of claim 14, wherein performing the response-provoking operation uses a third imaging objective.

18. The method of claim 17, further comprising:
switching—in the third imaging objective for the first imaging objective along the first optical path to perform the response-provoking operation; and
after performing the response-provoking operation, switching-back the first imaging objective for the third imaging objective along the first optical path.

19. The method of claim 1, further comprising selecting the NBL organism out of a plurality of like NBL organisms based on at least one criterion.

20. The method of claim 19, wherein at least one criterion comprises a manner of movement.

21. The method of claim 1, further comprising automatically analyzing behavior of the NBL organism based on the tracking data collected via the first imaging objective.

22. The method of claim 1, wherein the NBL organism is a C. elegans nematode.

23. The method of claim 1, wherein the NBL organism is a zebrafish larvae.

24. The method of claim 1, wherein the NBL organism is supported by a stage and the first and second imaging objectives are located on opposite sides of the stage.

25. A machine-readable storage medium containing machine-executable instructions for performing the method of claim 1.

26. A microscope system, comprising:
hardware for performing the method of claim 1; and
the machine-readable storage medium of claim 25 for controlling the hardware to allow a user to perform the method.

27. The microscope system of claim 26, wherein the hardware comprises:
a first imaging body that provides a first optical microscope light path for the first imaging objective; and
a second imaging body that provides a second optical microscope light path for the second imaging objective.

28. The microscope system of claim 26, wherein the first and second imaging bodies are parts of an optical microscope.

29. The microscope system of claim 26, wherein the first imaging body is part of a first optical microscope, and the second imaging body is part of a second optical microscope.

30. The microscope system of claim 26, wherein the first optical microscope light path in one of an inverted light path and an upright light path, and the second optical microscope light path is the other of the inverted light path and the upright light path.
